(19) United States
(12) Patent Application Publication
(10) Pub. No.: US 2007/0179365 A1
(43) Pub. Date: Aug. 2, 2007

(54) Method for monitoring body fluids

(75) Inventors: Gabriel Bitton, Jerusalem (IL); Benny Pesach, Rosh-Ha'ayin (IL); Ron Nagar, Tel-Aviv (IL)

Correspondence Address: WOLF, BLOCK, SCHORR & SOLIS-COHEN LLP, 250 PARK AVENUE, NEW YORK, NY, 10177, US

(73) Assignee: Glucon Inc., Boulder, CO (US)

(21) Appl. No.: 11/345,348

(22) Filed: Jan. 31, 2006

(51) Int. Cl. A61B 5/00; A61B 8/00

(52) U.S. Cl. 600/310; 600/449

(57) ABSTRACT

A method of monitoring at least one fluid compartment of a body comprising: illuminating the skin with light that stimulates photoacoustic waves in the skin; and using the photoacoustic waves to measure change in the volume of the fluid compartment.

### FIELD OF THE INVENTION

The invention relates to non-invasive in-vivo methods and apparatus for monitoring fluid compartments in the body.

### BACKGROUND OF THE INVENTION

The total liquid content of the body, conventionally referred to as total body water (TBW), is considered to be comprised in two “fluid compartments”, an intracellular fluid (ICF) compartment and an extracellular fluid (ECF) compartment. The ICF comprises the aggregate of fluids maintained within the body cells. The ECF comprises the interstitial fluid (ISF) that surrounds and bathes the body cells and the intravascular fluid (IVF), i.e. blood, carried by the vascular system. The ISF and the blood are “sub-compartments” of the ECF compartment. For convenience, compartments and sub-compartments are referred to generically as compartments.

The healthy body tends to maintain relatively stable, normative, ratios between the volumes of its various fluid compartments and equilibrium between their osmolarities. The body's ability to mediate stress, the possible failure or malfunction of a bodily process and the progress of a therapeutic intervention can be monitored by monitoring and detecting deviations of fluid compartment volumes and/or osmolarities and/or ratios of compartment volumes. For example, the onset and development of edema, the abnormal accumulation of interstitial fluid, which may result from any of many various conditions such as lymphedema, congestive heart failure, obesity, diseased leg veins, kidney disease, cirrhosis of the liver, anemia, and severe malnutrition, can be monitored by monitoring the volume of the interstitial fluid, ISF. Hypo- or hypervolaemia can be monitored by monitoring the volume of the blood.

It is known that the skin, and in particular the corium or dermis of the skin, is a major repository of body water, containing as much as 17% of the body's ISF, and that changes in thickness of the skin and/or dermis are correlated with changes in the volume of ISF. J. Schumacher et al in “Measurement of Peripheral Tissue Thickness by Ultrasound During The Perioperative Period”, British Journal of Anesthesia 82(4); 1999; pp-641-643 describe experiments showing that changes in thickness of skin on the forehead of a patient due to fluid depletion and fluid replacement during surgery were detectable using ultrasound. Change in the patient's blood volume during surgery was monitored to determine blood loss by assuming a constant erythrocyte volume (EV) and determining the packed red cell volume (PCV) of blood samples obtained from the patient by venepuncture. The determined PCV was corrected for the patient's body surface and blood volume, BV, was determined from an equation BV=EV/PCV.

W. Eichler et al in, “Changes of Interstitial Fluid Volume in Superficial Tissues Detected by a Miniature Ultrasound Device” J. Appl Physiol 89; 2000; pp 359-363 notes that “superficial tissue thickness can easily be determined by ultrasound techniques at body sites where the underlying bone provides a good backwall echo, such as the forehead or pretibial area.” The article describes using a miniature A-mode ultrasound device to evaluate skin thickness in a region of a patient's forehead in order to monitor the patient's ISF volume and thereby a fluid therapy. The article indicates that the described ultrasound methods and devices provide “an alternative to more invasive methods of fluid therapy monitoring”.

Mathematical models of water shift between compartments due to osmolarity change in one of the compartments are described in an article by C. C. Gyenge, et al; “Transport of Fluid and Solutes in the Body I. Formulation of a mathematical model”; Am J Physiol Heart Circ Physiol 277: H1215-H1227, 1999 and in an article in the same journal by the same authors entitled “Transport of Fluid and Solutes in the Body II. Model Validation and Implications”; Am J Physiol Heart Circ Physiol 277: H1228-H1240, 1999. The disclosure of the above cited articles are incorporated herein by reference.

### SUMMARY OF THE INVENTION

An aspect of some embodiments of the present invention relates to providing improved methods and apparatus for non-invasively monitoring volume changes in fluid compartments in the body.

An aspect of some embodiments of the invention relates to employing a photoacoustic effect to monitor volume changes of fluid compartments of the body.

In accordance with an aspect of an embodiment of the invention, a photoacoustic effect is used to provide measures of thickness of the skin and/or a layer or layers therein. The thickness of the skin and/or a skin layer and changes therein is used to monitor changes in the volume of ISF.

In accordance with an aspect of an embodiment of the invention, a photoacoustic effect is used to assay a marker substance, or determine a function of a marker substance, in a fluid compartment of the body. The concentration or function of the marker substance concentration is used to monitor changes in the volume of the fluid compartment.

A fluid compartment marker substance, also referred to as a “marker”, is a substance whose total quantity in the fluid compartment is substantially constant during a period of time for which it is used to measure changes in the fluid compartment's volume. As a result, a measure of changes in concentration of the marker or a function of the marker's concentration may be used to determine changes in the fluid compartment volume. For convenience, hereinafter, concentration of a marker and a function thereof are referred to generically as “concentration” of the marker. In an embodiment of the invention, the fluid compartment is the intravascular fluid, IVF, i.e. “blood”. Optionally, a marker substance for the IVF or blood is hemoglobin (Hb)and/or hematocrit (Hct) (i.e. packed red cell volume (PCV)).

In an embodiment of the invention, a body fluid monitor (BFM) comprises at least one light source that provides light to stimulate photoacoustic waves in the skin and in blood and at least one acoustic transducer that senses and generates signals responsive to the photoacoustic waves. Optionally, the at least one light source provides light at least one wavelength that is relatively strongly absorbed by ISF and/or components of the ISF and/or light at least one wavelength that is relatively strongly absorbed by a marker in the blood. In an embodiment of the invention the wavelength of light that is relatively strongly absorbed and/or scattered by ISF is a wavelength, such as 1440 nm, at which light is relatively strongly absorbed by water. Optionally, the marker in blood is hemoglobin and the wavelength of light that is relatively strongly absorbed by the marker, hemoglobin, is 800 nm.

To monitor changes in the volume of the ISF and the blood, the BFM is placed on the skin and the at least one light source is controlled to transmit light that illuminates and stimulates photoacoustic waves in the skin and blood in a blood vessel in and/or below the skin. The signals are processed to determine which are generated responsive to waves that originate in the skin and/or at boundaries of layers in the skin and which are responsive to waves that originate in the blood vessel. The signals that originate in the skin and/or skin boundaries are used to provide a measure of skin thickness and/or changes therein, which in turn is used to monitor the volume of the ISF.

For example, generally, photoacoustic signals that originate in or near boundaries of layers in the skin are particularly strong and these relatively strong signals may be used to identify the boundaries, relative distances between the boundaries and/or changes in the distances. Absolute distances between boundaries of the layers may be determined responsive to an assumed or measured speed of sound in the skin. The distances and/or changes therein are used to monitor skin thickness and/or changes therein and thereby ISF volume changes. The signals that originate in blood in the blood vessel are used to assay a marker in the blood and the assay used to monitor the blood volume.

In an embodiment of the invention, a BFM monitors distance of a blood vessel below the surface of the skin to monitor changes in skin thickness and thereby ISF volume changes. Optionally, the depth of the blood vessel below the skin is determined responsive to photoacoustic waves stimulated in the blood vessel. Optionally, the BFM transmits ultrasound waves into the skin and reflections of acoustic energy from the transmitted sound waves are used to monitor changes in depth of the blood vessel.

There is therefore provided in accordance with an embodiment of the invention a method of monitoring at least one fluid compartment of a body comprising: illuminating the skin with light that stimulates photoacoustic waves in the skin; and using the photoacoustic waves to measure change in the volume of the fluid compartment. Optionally, using the photoacoustic waves comprises using the photoacoustic waves to determine change in thickness of a layer or layers in the skin. Additionally or alternatively, using the photoacoustic waves optionally comprises using the waves to determine change in the concentration of a marker in a fluid compartment of the at least one fluid compartment.

In an embodiment of the invention, the at least one compartment comprises the interstitial fluid (ISF) compartment.

Optionally, illuminating the skin comprises illuminating the skin with light that is relatively strongly absorbed by water. Optionally, using the photoacoustic waves comprises using the photoacoustic waves to determine change in thickness of the skin and/or a layer therein. Optionally, using the photoacoustic waves comprises using the waves to determine change in distance at which a blood vessel is located in or beneath the skin.

In an embodiment of the invention, the at least one compartment comprises the blood. Optionally, using the photoacoustic waves comprises using the waves to assay a marker in the blood. Optionally, the marker is at least one of hemoglobin (Hb), or packed red cell volume (PCV). Optionally, illuminating the skin comprises illuminating the skin with light that is relatively strongly absorbed by hemoglobin.

In an embodiment of the invention, the at least one compartment comprises two compartments. Optionally, the two compartments comprise the ISF compartment and the blood. Optionally, determining change in the ISF compartment volume comprises using the photoacoustic waves to determine change in distance at which a blood vessel is located in or beneath the skin. Optionally, determining change in the volume of blood comprises using the photoacoustic waves to assay a marker in the blood.

There is further provided in accordance with an embodiment of the invention a method of determining change in thickness of the skin comprising: illuminating the skin with light that stimulates photoacoustic waves in the skin; and using the photoacoustic waves to determine change in the thickness.

There is further provided in accordance with an embodiment of the invention a method of determining change in thickness of the skin comprising: illuminating the skin with light that stimulates photoacoustic waves in a blood vessel in or below the skin; using the photoacoustic waves to determine change in distance of the blood vessel below the skin surface; and using change in the distance to determine change in skin thickness.

There is further provided in accordance with an embodiment of the invention a method of determining changes in thickness of the skin comprising: transmitting ultrasound that is reflected from a blood vessel in or below the skin; using the reflected ultrasound to determine change in distance at which the blood vessel is located beneath the skin surface; and using changes in the distance to determine changes in skin thickness.

### BRIEF DESCRIPTION OF FIGURES

Non-limiting examples of embodiments of the invention are described below with reference to figures attached hereto. In the figures, identical structures, elements or parts that appear in more than one figure are generally labeled with the same numeral in all the figures in which they appear. Dimensions of components and features shown in the figures are chosen for convenience and clarity of presentation and are not necessarily shown to scale. The figures are listed below.

FIG. 1 schematically shows a body fluid monitor, BFM, being used to monitor the volume of ISF and blood of a patient's body in accordance with an embodiment of the present invention; and

FIG. 2 schematically shows signals generated by the BFM shown in FIG. 1 that are used to monitor the ISF and blood, in accordance with an embodiment of the invention.

### DETAILED DESCRIPTION OF EXEMPLARY EMBODIMENTS

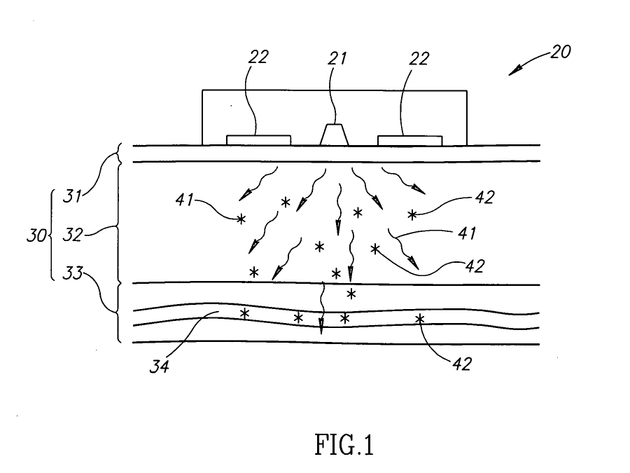

FIG. 1 schematically shows a BFM 20 located on skin 30 of a patient being operated to monitor the patient's ISF and blood, in accordance with an embodiment of the invention. Skin 30 comprises the epidermis 31, the corium or dermis 32 and the subcutis 33. Dermis 32 is a tough elastic layer containing fibrous tissue interlaced with elastic fibers. Most of the ISF that the skin contains is stored in the corium. Subcutis 33 comprises mainly loose fibrous connective tissue and fat cells laced with blood vessels. A blood vessel 34 schematically represents the blood vessels in subcutis 33. BFM 20 comprises at least one light source 21 that provides pulses of light that stimulate photoacoustic waves in the ISF in corium 32 and in blood in blood vessel 34 and at least one acoustic transducer 22 that generates signals responsive to photoacoustic waves stimulated by the light pulses.

Light in a light pulse provided by light source 21 is schematically represented by wavy arrows 41 and the locations at which photoacoustic waves are stimulated by the light are schematically represented by asterisks 42. For convenience, the numeral 42 is also used to reference the photoacoustic waves stimulated by light 41 at locations 42. Optionally, light source 21 provides light 41 at a wavelength, such as 1440 nm, that is relatively strongly absorbed by water to stimulate photoacoustic waves 42 in the ISF contained in corium 32. In general, at 1440 nm, light is so strongly absorbed by water that very little of the light reaches blood vessel 34 and light at this wavelength is relatively ineffective at stimulating photoacoustic waves in blood vessel 34. Optionally, light source 21 provides light at a wavelength, such as 810 nm, that is relatively strongly absorbed by hemoglobin to stimulate photoacoustic waves 42 in blood vessel 34. In some embodiments of the invention, light source 21 provides light 41 at a wavelength, such as 1650 nm, at which the light is satisfactorily effective in stimulating photoacoustic waves in both the ISF and in blood in blood vessel 34.

By way of example, BFM 20 is shown comprising one light source 21 flanked by two acoustic transducers 22. Any configuration of light sources and acoustic transducers suitable for generating photoacoustic waves in skin 30 and sensing the photoacoustic waves may be used in the practice of the present invention. Photoacoustic sensors, such as those shown and described in PCT Publication WO2005/068973, the disclosure of which is incorporated herein by reference, are optionally used in the practice of the present invention.

In accordance with an embodiment of the invention, BFM 20 periodically illuminates skin 30 with a pulse of light 41 and processes signals generated by at least one transducer 22 responsive to photoacoustic waves 42 stimulated by the light to determine thickness of the skin and assay hemoglobin in blood vessel 34. Changes in the determined thickness and assay are used to monitor changes in the volumes of the ISF and blood.

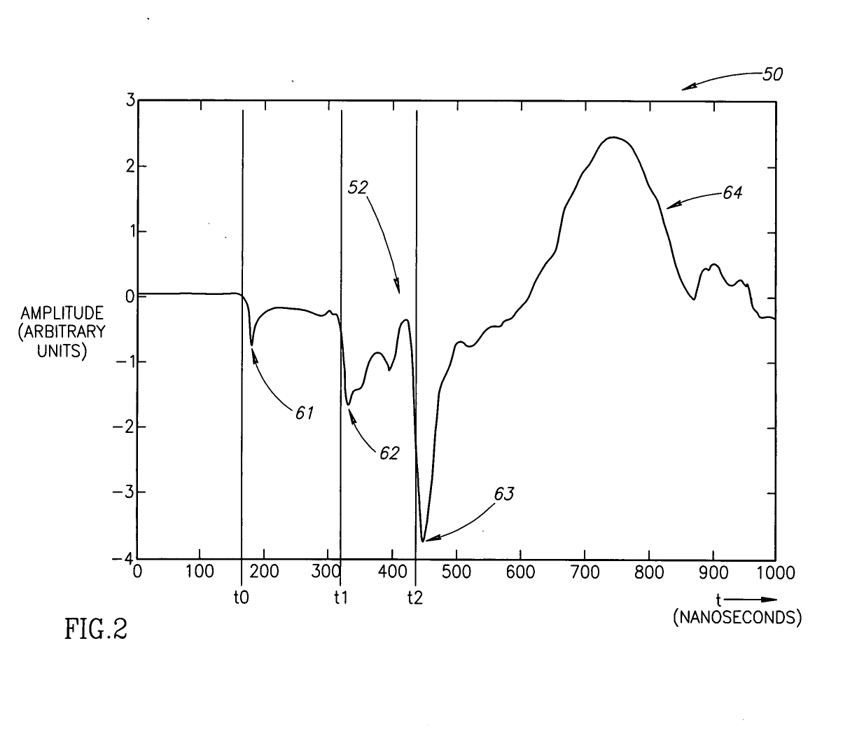

FIG. 2 schematically shows a graph 50 of a signal generated by at least one acoustic transducer 22 when BFM 20 illuminates skin 30 with a pulse of light at a wavelength at which light is relatively strongly absorbed by hemoglobin. A curve 52 shows the amplitude of the signal, measured in arbitrary units along the ordinate of graph 50, as a function of time, which is indicated in nanoseconds along the abscissa of the graph.

A first negative peak 61 (polarity of peak 61 and other peaks is arbitrary and a function of the configuration of at least one transducer 22) in signal 52 is generated by at least one transducer 22 in response to light reflected from the surface of skin 30 that is incident on the at least one transducer. The light causes local heating in a region of the surface of at least one transducer 22 that produces sound waves in the transducer which generate negative peak 61. The negative peak begins at a time tₒ substantially simultaneous with a time at which at least one light source 21 transmits the pulse of light 41 which illuminates skin 30.

A second negative peak 62 in signal 52 is generated and begins at a time t₁ in response to photoacoustic waves 42 stimulated in skin 30 and in particular in corium 32 as a result of absorption of light 41 by the skin. The photoacoustic waves are generated following a short time delay’, i.e. a “release delay”, after energy from light 41 is absorbed substantially at time tₒ by skin 30. Time t₁ follows time tₒ by a time delay substantially equal to the release delay time and a transmit time delay that is substantially equal to a time it takes sound to travel from the boundary between epidermis 31 and corium 32 to at least one detector 22.

A third negative peak 63 is generated in response to photoacoustic waves 42 stimulated in blood in blood vessel 34 as a result of absorption of light 41 by hemoglobin in the blood and begins at a time t₂. Whereas, photoacoustic waves in skin 30 and blood vessel 34 are generated substantially at a same time tₒ, time t₂ is delayed with respect to time t₁ by the release delay and a transmit time of photoacoustic waves from the blood vessel to transducers 22. (Since transducers 22 contact the skin, t₁ is determined substantially by the release delay and is substantially independent of transit time.) The relatively large positive pulse 64 that follows pulse 63 is generated by at least one detector 22 in response to decay of pressure from photoacoustic waves 42 that generated pulse 63.

Thickness D of the skin is optionally determined responsive to a time difference between negative peaks 62 and 61. Optionally, the difference is a time delay (t₂-t₁) between the onset time of negative peak 63 and the onset time of negative peak 62 and D is determined in accordance with an expression D=(t₂-t₁)c where c is a known speed of sound in skin. D and changes ΔD therein are used to monitor the volume, “V_{ISF}”, and changes ΔV_{ISF} therein of the ISF using known relationships between skin thickness and volume of ISF. Optionally the relationships between changes in skin thickness and changes in volume of ISF are determined responsive to theoretical and/or empirical studies, for example from studies by J. Schumacher et al, W. Eichler et al, and C. C. Gyenge, et al noted above and/or, for a particular patient, in a calibration procedure performed on the patient. Optionally, changes in thickness D of skin 30 is assumed to be proportional to changes in the volume of ISF and ΔD/D=βΔV_{ISF}/V_{ISF}, where β is a constant determined in a calibration procedure.

The amplitude and shape of negative peak 63 is a function of the concentration of hemoglobin in blood in blood vessel 34 and, in an embodiment of the invention, the hemoglobin is assayed responsive to the amplitude and/or shape of the peak 63 and/or a time integral of the amplitude of the peak. Optionally signal 52 and peak 63 are processed to assay the hemoglobin using methods described in an article by A. A. Oraevsky et al entitled “Determination of Tissue Optical Properties by Piezoelectric Detection of Laser-Induced Stress Waves”; SPIE Vol. 1882 Laser-Tissue Interaction IV (1993); pp 86-98, the disclosure of which is incorporated herein by reference. Since hemoglobin is substantially confined to blood, it is a marker for the volume of blood in the vascular system and relative changes in blood hemoglobin concentration are negatives of corresponding relative changes in blood volume. In particular, if V_{b} is the volume of the intravascular fluid, i.e. the blood, and ρₕ is the concentration of hemoglobin in the blood, then ΔV_{b}/V_{b}=−Δρₕ/ρₕ. And, in accordance with an embodiment of the invention, changes in the hemoglobin assay determined responsive to photoacoustic waves stimulated by light from at least one light source 21 are used as measures of changes in the blood volume and to monitor changes in the blood volume.

It is noted that whereas signal 52 is assumed to be generated responsive to photoacoustic waves stimulated by light at a wavelength of light that is relatively strongly absorbed by hemoglobin, other wavelengths can be used. For example, light source 21 may illuminate skin 30 with light that is relatively highly absorbed and/or scattered by water. Light at such a wavelength stimulates relatively strong photoacoustic waves in corium 32 and allows BFM 20 to provide signals that are relatively sensitive, not only to the boundaries of the corium but also to the concentration of water in the corium. The signals may be processed to provide relatively accurate measures of the thickness of the corium 32 and skin 30 and an assay of water in the corium. In accordance with an embodiment of the invention, the different measures, i.e. thickness and water assay, are combined and/or compared to provide a measure of skin thickness and/or changes therein having improved accuracy.

In some embodiments of the invention, acoustic transducers 22 are used not only to sense photoacoustic waves but are also used to transmit ultrasound into skin 30 and sense and generate signals responsive to ultrasound waves reflected by features in the skin. The signals generated responsive to the ultrasound reflections are processed to provide measures of thickness of skin 30 and/or its layers.

For example, a distance at which blood vessel 34 in subcutis 33 or a blood vessel under the subcutis is located below epidermis 31 is determined substantially by the thickness of the dermis 32. Reflections of ultrasound from blood vessel 34 or a blood vessel beneath subcutis 33 are used in accordance with an embodiment of the invention to determine changes in the thickness of dermis 32 and thereby changes in the volume of ISF. Optionally, the ultrasound measures of skin thickness are combined with photoacoustic measures of skin thickness to provide a measure of skin thickness and/or changes therein having improved accuracy.

The inventors have noted that measurements of skin thickness and/or changes therein determined responsive to reflections of ultrasound from bone underlying the skin are relatively sensitive to motion of the skin surface relative to the bone. For example, muscle motion and pressure on the skin in an area at which the skin thickness is measured will in general tend to change the distance of the skin surface from the underlying bone and thereby influence skin thickness measurements. Distance of a blood vessel under the skin is relatively insensitive to such motion. As a result, skin thickness and/or change therein determined responsive to ultrasound reflections from blood vessels in accordance with an embodiment of the invention, tends to be more reliable and accurate than skin thickness and/or skin thickness change determined responsive to bone-reflected ultrasound. It is noted that photoacoustic waves generated by light absorbed by a marker in the blood can also be used, in accordance with an embodiment of the invention, to determine a distance at which a blood vessel is located beneath the skin and therefrom a skin thickness.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art. The scope of the invention is limited only by the following claims.

1. A method of monitoring at least one fluid compartment of a body comprising:
illuminating the skin with light that stimulates photoacoustic waves in the skin; and
using the photoacoustic waves to measure change in the volume of the fluid compartment.

2. A method according to claim 1 wherein using the photoacoustic waves comprises using the photoacoustic waves to determine change in thickness of a layer or layers in the skin.

3. A method according to claim 1 wherein using the photoacoustic waves comprises using the waves to determine change in the concentration of a marker in a fluid compartment of the at least one fluid compartment.

4. A method according to claim 1 wherein the at least one compartment comprises the interstitial fluid (ISF) compartment.

5. A method according to claim 4 wherein illuminating the skin comprises illuminating the skin with light that is relatively strongly absorbed by water.

6. A method according to claim 4 wherein using the photoacoustic waves comprises using the photoacoustic waves to determine change in thickness of the skin and/or a layer therein.

7. A method according to claim 6 wherein using the photoacoustic waves comprises using the waves to determine change in distance at which a blood vessel is located in or beneath the skin.

8. A method according to claim 1 wherein the at least one compartment comprises the blood.

9. A method according to claim 8 wherein using the photoacoustic waves comprises using the waves to assay a marker in the blood.

10. A method according to claim 9 wherein the marker is at least one of hemoglobin (Hb), or packed red cell volume (PCV).

11. A method according to claim 8 wherein illuminating the skin comprises illuminating the skin with light that is relatively strongly absorbed by hemoglobin.

12. A method according to claim 1 wherein the at least one compartment comprises two compartments.

13. A method according to claim 12 wherein the two compartments comprise the ISF compartment and the blood.

14. A method according to claim 13 wherein determining change in the ISF compartment volume comprises using the photoacoustic waves to determine change in distance at which a blood vessel is located in or beneath the skin.

15. A method according to claim 14 wherein determining change in the volume of blood comprises using the photoacoustic waves to assay a marker in the blood.

16. A method of determining change in thickness of the skin comprising:
illuminating the skin with light that stimulates photoacoustic waves in the skin; and
using the photoacoustic waves to determine change in the thickness.

17. A method of determining change in thickness of the skin comprising:
illuminating the skin with light that stimulates photoacoustic waves in a blood vessel in or below the skin;
using the photoacoustic waves to determine change in distance of the blood vessel below the skin surface; and
using change in the distance to determine change in skin thickness.

18. A method of determining changes in thickness of the skin comprising:
transmitting ultrasound that is reflected from a blood vessel in or below the skin;
using the reflected ultrasound to determine change in distance at which the blood vessel is located beneath the skin surface; and
using changes in the distance to determine changes in skin thickness.
